(19) United States
(12) Patent Application Publication
(10) Pub. No.: US 2016/0166249 A1
(43) Pub. Date: Jun. 16, 2016

(54) STAPLING DEVICE WITH DISTALLY LOCATED HYDRAULIC DRIVE- RECIPROCALLY OPERATED SYSTEM AND METHOD

(71) Applicant: Cardica, Inc., Redwood City, CA (US)

(72) Inventor: Bryan D. Knodel, Flagstaff, AZ (US)

(21) Appl. No.: 14/793,592

(22) Filed: Jul. 7, 2015

Related U.S. Application Data
(60) Provisional application No. 62/091,171, filed on Dec. 12, 2014.

(51) Int. Cl. A61B 17/068; A61B 17/10

(52) U.S. Cl. CPC A61B 17/068; A61B 17/105

(57) ABSTRACT

A surgical stapling device is configured for use in open and/or laparoscopic surgical procedures. The device includes a handle assembly, a shaft assembly coupled to the handle assembly, and an end-effector coupled to the shaft assembly. The end-effector comprises of a jaw assembly configured to clamp, staple, and/or cut a target tissue. The handle assembly comprises of a trigger member that can activate a control member to close the jaw assembly to clamp, staple, and/or cut the target tissue. The end-effector includes a reciprocating hydraulic drive system to provide the power necessary to drive the deployment operations, such as deploying staples and cutting tissue.

### FIELD OF THE INVENTION

The present invention relates generally to surgical devices, and more particularly to surgical stapling or clip applying systems. The application claims priority to Provisional application No. 62/091,171, which was filed on Dec. 12, 2014.

### BACKGROUND

Surgical stapling devices, such as endocutters, typically staple and cut tissue to transect that tissue while leaving the cut ends of that tissue hemostatic. More advanced surgical stapling devices typically have end-effectors that are small enough in diameter so that they can be used in minimally invasive surgical procedures where access to a surgical site is obtained through a trocar, port, or small incision in the body of a patient. A typical stapling device holds a disposable single-use cartridge with several rows of staples, and includes an anvil to oppose the staples as the staples are deployed from the cartridge. During operations, the surgeon inserts the stapling device through an opening in the body (typically using a trocar), orients the end of the stapling device around the tissue to be transected, and compresses the anvil and cartridge together to clamp that tissue. Then, a row or rows of staples are deployed on either side of the transection line, and a blade is advanced along the transection line to divide the tissue.

During actuation of the endocutter, the stapling device fires all of the staples in the single-use disposable cartridge. In order to deploy more staples, the endocutter must be moved away from the surgical site and removed from within the patient. The spent cartridge is removed from the endocutter and replaced by a new cartridge. The endocutter is then reinserted into the patient for further staple deployment.

Accordingly, it would be desirable to miniaturize the components within the end-effector of the stapling device to allow for greater operability within a small space.

### SUMMARY OF THE INVENTION

A surgical stapling device is configured for use in open and/or laparoscopic surgical procedures. The device includes a handle assembly, a shaft assembly coupled to the handle assembly, and an end-effector coupled to the shaft assembly. The end-effector comprises of a jaw assembly configured to clamp, staple, and/or cut a target tissue. The handle assembly comprises of a trigger member that can activate a clamp control member to close the jaw assembly on the target tissue. The stapling device includes a reciprocating hydraulic drive system within the end-effector to provide direct driving of a deployment assembly member to deploy staples.

A surgical stapling device is configured for use in open and/or laparoscopic surgical procedures. The device includes a handle assembly, a shaft assembly coupled to the handle assembly, and an end-effector coupled to the shaft assembly. The end-effector comprises of a jaw assembly configured to clamp, staple, and/or cut a target tissue. The handle assembly comprises of a trigger member that can activate a clamp control member to close the jaw assembly on the target tissue. The stapling device includes a reciprocating hydraulic drive system within the end-effector to provide direct driving of a deployment assembly member to deploy staples. The stapling device further comprises a shaft member coupled to the end-effector, wherein the shaft member includes a flexible segment to allow articulation of the end-effector. Hydraulic supply lines or hoses are routed from within the shaft member to the end-effector to operate the reciprocating drive system within the end-effector.

The reciprocating hydraulic system includes a piston member. The piston member directly drives the deployment assembly member to deploy staples. An actuation member is disposed between the piston and the deployment assembly member. The actuation member may be substantially rigid or stiff. The piston member drives the deployment assembly member through the actuation member.

The actuation member my include engagement features to engage with the deployment assembly member during a drive stroke of a reciprocating cycle of the reciprocating hydraulic drive system and to disengage with the deployment assembly member during a recovery stroke of the reciprocating cycle of the reciprocating hydraulic drive system.

The deployment assembly member includes a locking element to engage with at least one of the engagement features of the actuation member during the drive stroke of the reciprocating cycle of the reciprocating hydraulic drive system and to disengage with the at least one of the engagement features of the actuation member during the recovery stroke of the reciprocating cycle of the reciprocating hydraulic drive system.

The reciprocating hydraulic drive system includes a through-channel to provide a pass-through from the shaft member of the stapling device to the end-effector of the stapling device. A control member originating from the proximal portion of the stapling device disposed through the through-channel to couple with the deployment assembly member, wherein the control member is configured to reset the deployment assembly member. The control member resets the deployment assembly member after deployment of the staples by the deployment assembly member.

### BRIEF DESCRIPTION OF THE DRAWINGS

The present invention will be readily understood by the following detailed description, taken in conjunction with accompanying drawings, illustrating by way of examples of the embodiments of the invention. The figures are merely exemplary and not limiting. The objects and elements in the drawings are not necessarily drawn to scale, proportion, precise orientation or positional relationships; instead, emphasis is focused on illustrating the principles of the invention. Descriptive terms such as “upper,” “lower,” “upward,” “downward”, “forward”, “backward”, and the like are intended for the convenience of the reader and refer to the orientation and/or motion of parts as illustrated and described; they do not necessarily limit the orientation or operation of the features, aspects, or embodiments of the invention. The drawings illustrate the design and utility of various features, aspects, or embodiments of the present invention, in which like element are typically referred to by like reference symbols or numerals. The drawings, however, depict the features, aspects, or embodiments of the invention, and should not be taken as limiting in their scope. With this understanding, the features, aspects, or embodiments of the invention will be described and explained with specificity and details through the use of the accompanying drawings in which:

FIG. 1A illustrates an example of a surgical stapling device, in accordance with features, aspects or embodiments of the present invention.

FIG. 1B illustrates the distal portion of the surgical stapling device which includes an articulation segment that allows an end-effector of the surgical stapling device to articulate, in accordance with features, aspects or embodiments of the present invention.

FIG. 2A illustrates a joint portion of the end-effector of the surgical stapling device including some of the control elements that control the operation of the end-effector, in accordance with features, aspects or embodiments of the present invention.

FIG. 2B illustrates a partially exposed joint portion of the end-effector of the surgical stapling device to further show some of the control elements that control the operation of the end-effector, in accordance with features, aspects or embodiments of the present invention.

FIG. 3 illustrates an isometric view of a hydraulic drive system that is housed distally near the joint portion of the end-effector of the stapling device, in accordance with features, aspects or embodiments of the present invention.

FIG. 4A illustrates a side view of the joint portion of the end-effector with the hydraulic drive system housed distally of the joint connection, in accordance with features, aspects or embodiments of the present invention.

FIG. 4B illustrates an exposed view of the joint portion of the end-effector with the hydraulic drive system housed distally of the joint connection, in accordance with features, aspects or embodiments of the present invention.

FIG. 5A illustrates an exposed view of the hydraulic drive system with a drive or actuation member coupled to a piston member of the hydraulic drive system, wherein within the hydraulic drive system the piston member is illustrated in a first position, in accordance with features, aspects or embodiments of the present invention.

FIG. 5B illustrates an exposed view of the hydraulic drive system with a drive or actuation member coupled to a piston member of the hydraulic drive system, wherein within the hydraulic drive system the piston member is illustrated in a second position, in accordance with features, aspects or embodiments of the present invention.

FIG. 6A illustrates (a side view) that the hydraulic drive system through the drive or actuation member is coupled to a knife member and a wedge assembly, wherein the drive or actuation member is configured to advance distally said knife member and said wedge assembly to deploy staples and cut tissue, in accordance with features, aspects or embodiments of the present invention.

FIG. 6B illustrates (a top view) that the hydraulic drive system through the drive or actuation member is coupled to a knife member and a wedge assembly, wherein the drive or actuation member is configured to advance distally said knife member and said wedge assembly to deploy staples and cut tissue, in accordance with features, aspects or embodiments of the present invention.

FIG. 7A illustrates the advancement of the drive or actuation member coupled to hydraulic drive system which advances distally the wedge assembly and knife member of the surgical stapling device to deploy staples and cut tissue, in accordance with features, aspects or embodiments of the present invention.

FIGS. 7B and 7C illustrate (top views) the reciprocating movement of the drive or actuation member driven by the piston member to advance distally the wedge assembly and knife member of the surgical stapling device to deploy staples and cut tissue, in accordance with features, aspects or embodiments of the present invention.

FIGS. 8A and 8B illustrate (side views) the reciprocating movement of the drive or actuation member driven by the piston member to advance distally the wedge assembly and knife member of the surgical stapling device to deploy staples and cut tissue, in accordance with features, aspects or embodiments of the present invention.

FIG. 9A illustrates the wedge assembly and the knife member being retracted proximally to a first retracted position by a control element, wherein the control element is operated through a channel in the hydraulic drive system by control mechanisms in the stapling device, in accordance with features, aspects or embodiments of the present invention.

FIG. 9B illustrates the wedge assembly and the knife member being retracted proximally to a second retracted position by a control element, wherein the control element is operated through a channel in the hydraulic drive system by control mechanisms in the stapling device, in accordance with features, aspects or embodiments of the present invention.

FIG. 10A illustrates a close-up isometric view of the knife member and wedge assembly being retracted proximally by the control element, wherein a locking element allows the knife member and wedge assembly to be retracted proximally along the drive or actuation member, wherein the drive or actuation member includes features configured to engage the locking element when the drive or actuation member is operated to advance the knife and wedge assembly and the features disengage with the locking element when the knife member and wedge member are retracted, in accordance with features, aspects or embodiments of the present invention.

FIG. 10B illustrates a close-up side view of the knife member and wedge assembly being retracted proximally by the control element, wherein a locking element allows the knife member and wedge assembly to be retracted proximally along the drive or actuation member, wherein the drive or actuation member includes features configured to engage the locking element when the drive or actuation member is operated to advance the knife and wedge assembly and the features disengage with the locking element when the knife member and wedge member are retracted, in accordance with features, aspects or embodiments of the present invention.

FIG. 11A illustrates another close-up isometric view of the knife member and wedge assembly being retracted proximally by the control element, wherein a locking element allows the knife member and wedge assembly to be retracted proximally along the drive or actuation member, wherein the drive or actuation member includes features configured to engage the locking element when the drive or actuation member is operated to advance the knife and wedge assembly and the features disengage with the locking element when the knife member and wedge member are retracted, in accordance with features, aspects or embodiments of the present invention.

FIG. 11B illustrates another close-up side view of the knife member and wedge assembly being retracted proximally by the control element, wherein a locking element allows the knife member and wedge assembly to be retracted proximally along the drive or actuation member, wherein the drive or actuation member includes features configured to engage the locking element when the drive or actuation member is operated to advance the knife and wedge assembly and the features disengage with the locking element when the knife member and wedge member are retracted, in accordance with features, aspects or embodiments of the present invention.

FIG. 11C illustrates a top view of the knife member and wedge assembly being retracted proximally by the control element, wherein a locking element allows the knife member and wedge assembly to be retracted proximally along the drive or actuation member, wherein the drive or actuation member includes features configured to engage the locking element when the drive or actuation member is operated to advance the knife and wedge assembly and the features disengage with the locking element when the knife member and wedge member are retracted, in accordance with features, aspects or embodiments of the present invention.

### DETAILED DESCRIPTION

In the following detailed description, specific details are set forth in order to provide a thorough understanding of the present invention. However, it will be readily understood by those skilled in the art that the present invention may be practiced without these specific details. Alternatively, some of the well-known parts, components, hardware, methods of operations, and procedures may not be described in detail or elaborated so as to avoid obscuring the present invention; but, nevertheless, they are within the spirit and scope of the present invention.

As mentioned, surgical stapling devices, such as endocutters, typically staple and cut tissue to transect that tissue while leaving the cut ends of that tissue hemostatic. More advanced surgical stapling devices typically have end-effectors that are small enough in diameter so that they can be used in minimally invasive surgical procedures where access to a surgical site is obtained through a trocar, port, or small incision in the body of a patient. A typical stapling device holds a disposable single-use cartridge with several rows of staples, and includes an anvil to oppose the staples as the staples are deployed from the cartridge. During operations, the surgeon inserts the stapling device through an opening in the body (typically using a trocar), orients the end of the stapling device around the tissue to be transected, and compresses the anvil and cartridge together to clamp that tissue. Then, a row or rows of staples are deployed on either side of the transection line, and a blade is advanced along the transection line to divide the tissue.

As can be appreciated, it would be highly desirable to develop and implement miniaturized mechanisms and features that can drive or operate various functions of a surgical stapling device. Towards that end, this disclosure describes a stapling device with a distally located hydraulic drive system in accordance with features, aspects or embodiments of the present invention.

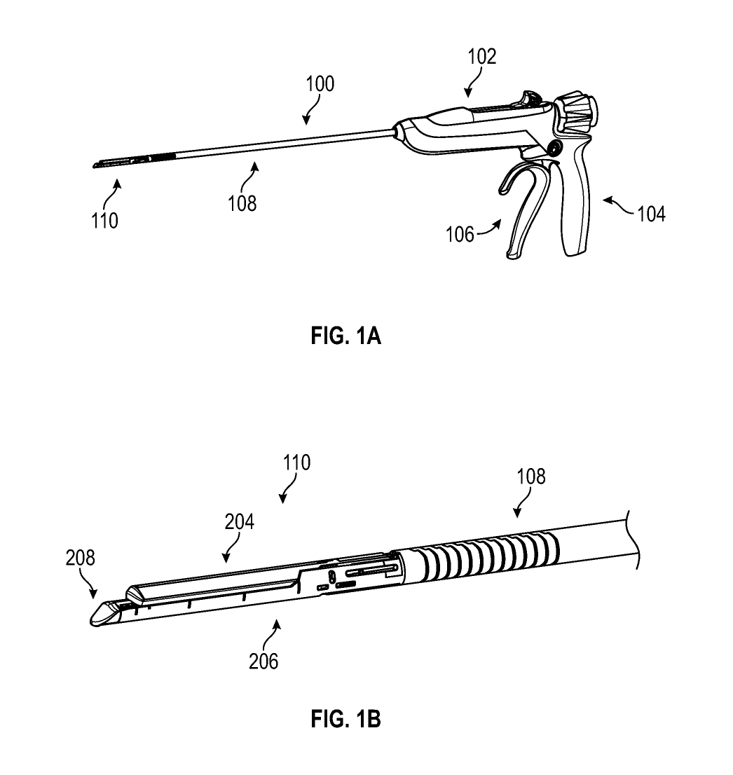

By way of example, FIG. 1A illustrates a surgical stapling device 100 in accordance with features, aspects and embodiments of the present invention. As illustrated, the surgical stapling device 100 includes a body portion 102, a handle portion 104, a trigger member 106, a shaft member 108, and an end-effector 110. FIG. 1B illustrates a close up view of the distal portion of the shaft member 108 along with the end-effector 110. As can be seen, the distal portion of the shaft member 108 may include a flexible segment or flexible region such that the shaft member 108 may be articulated. In some embodiments, the shaft member 108 may include a flexible section (as illustrated), and in some embodiments, the shaft member 108 may be a substantially rigid shaft. Further illustrated in FIG. 1B, the end-effector 110 may include jaw members such as an anvil member 204 and a staple holder channel member 206. The staple holder channel member 206 may be configured to hold a staple cartridge 208. The staple cartridge 208 may include staples and a cutting member for stapling and cutting tissue(s).

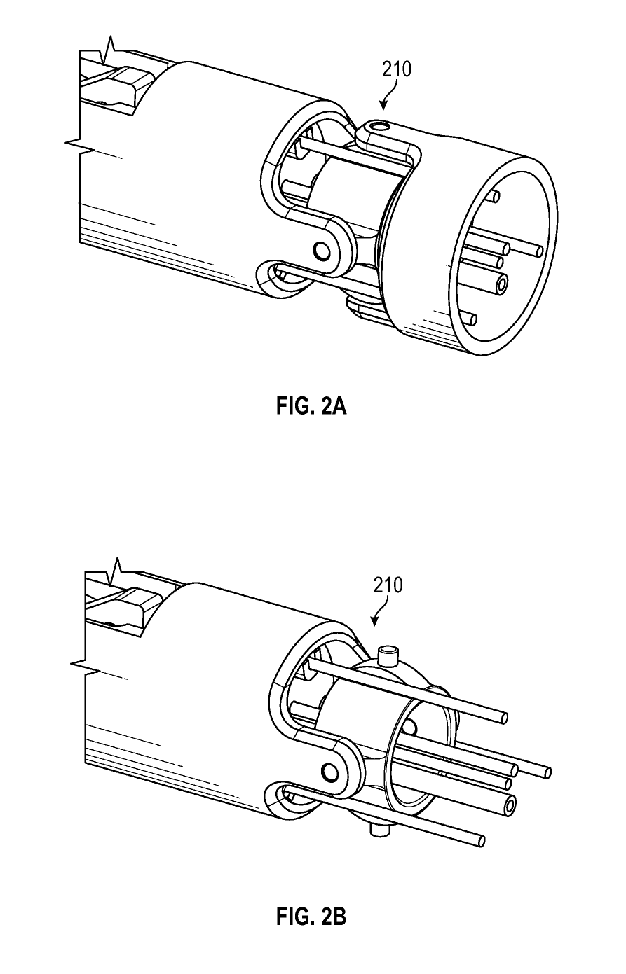

FIG. 2A and FIG. 2B illustrate a joint member 210 between the shaft member 108 and the end-effector 110 in accordance with features, aspects and embodiments of the present invention. As illustrated in FIG. 2A, the joint member 210 is a substantially flexible joint connecting the shaft member 108 and the end-effector 110. In addition, as illustrated in FIG. 2A and FIG. 2B, the joint member 210 allows a number of channels to be passed through from the shaft member 108 to the end-effector 110. These channels includes through channels for passing various surgical instruments, e.g., various optical scopes, grabbers, tweezers, catchers, ultra-sonic devices, RF devices, etc., from the proximal portion of the stapling device 100 to the distal portion of the stapling device. In addition, some of these channels may be flexible tubing for delivering hydraulic fluids for operating a hydraulic system (to be discussed in more details).

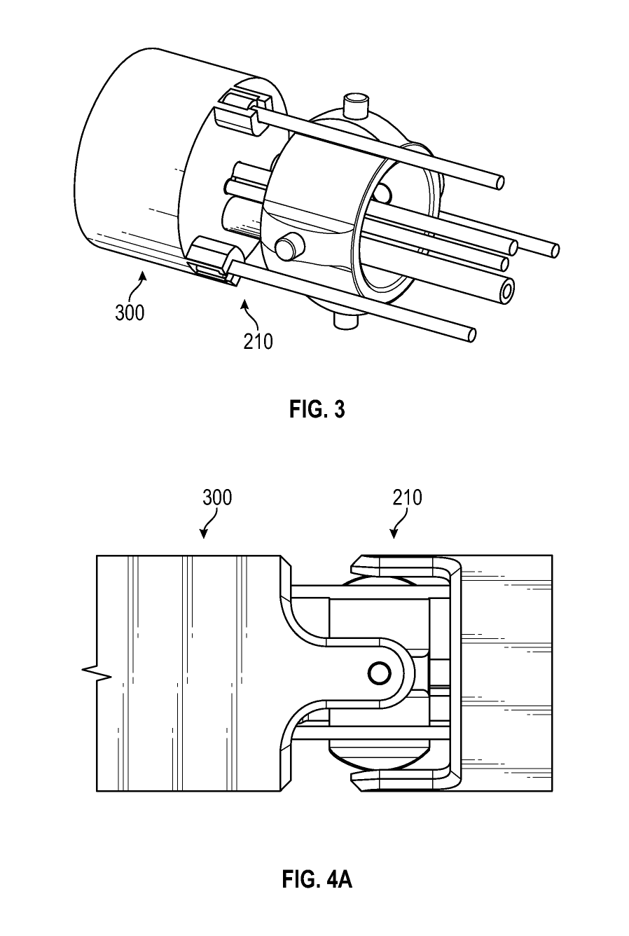

FIG. 3 illustrates an isometric view of a hydraulic drive system 300 that is housed or located distally of the joint member 210 and near the proximal portion of the end-effector 110. The hydraulic drive system 300 is configured to provide the necessary driving force to operate the deployment operations of the staple device 100. The hydraulic drive system as contained within the end-effector is configured to provide “direct drive input” for deployment operations of the stapling device. Such direct drive input provided by the hydraulic drive system enhances mechanical output advantages. In other words, the features, aspects, and embodiments of the present invention implements a “direct drive” system right near the very tip of the end-effector 110, where deployment operations are executed, to provide increased efficient operating or driving force necessary to performance various deployment operations of the stapling device 100.

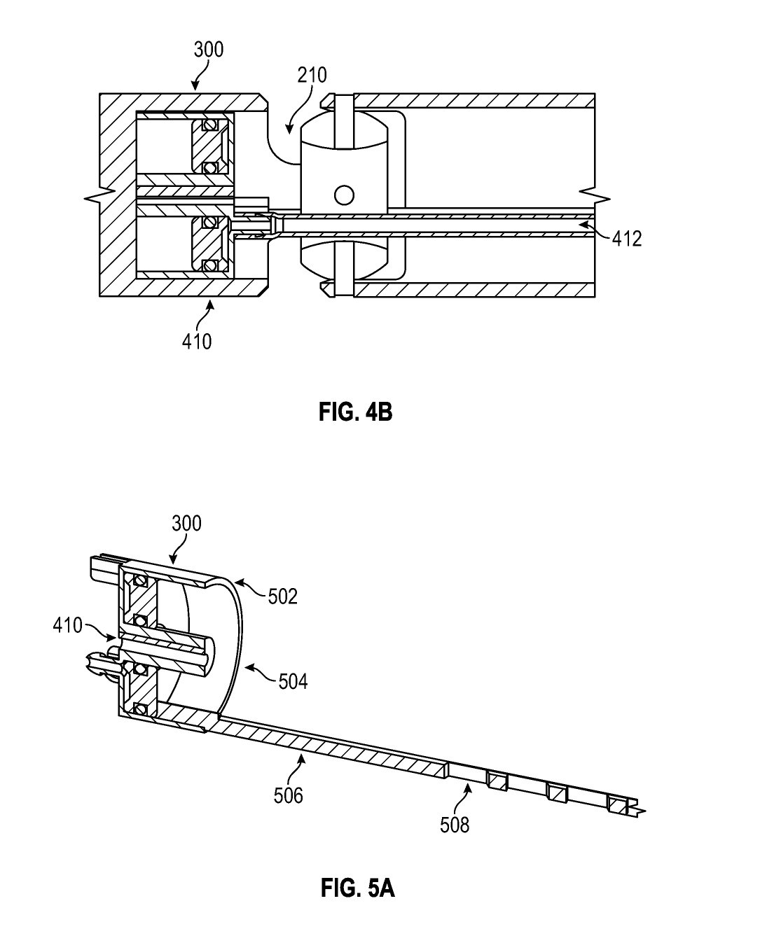

FIG. 4A and FIG. 4B illustrate side views of the joint member 210 connecting the shaft member 108 and the end-effector 110. In addition, the hydraulic drive system 300 is housed near the proximal portion of the end-effector 110 and distally to the joint member 210. FIG. 4A illustrates a side view of the joint portion of the end-effector with the hydraulic drive system 300. FIG. 4B illustrates an exposed view of the joint portion of the end-effector 110 with hydraulic drive system 300 housed distally of the joint connection. Also illustrated in FIG. 4B, a drive piston member 410 is operated by one or more hydraulic supply and/or return lines 412 to product reciprocating motions as the input power to execute deployment operations of the stapling device 100.

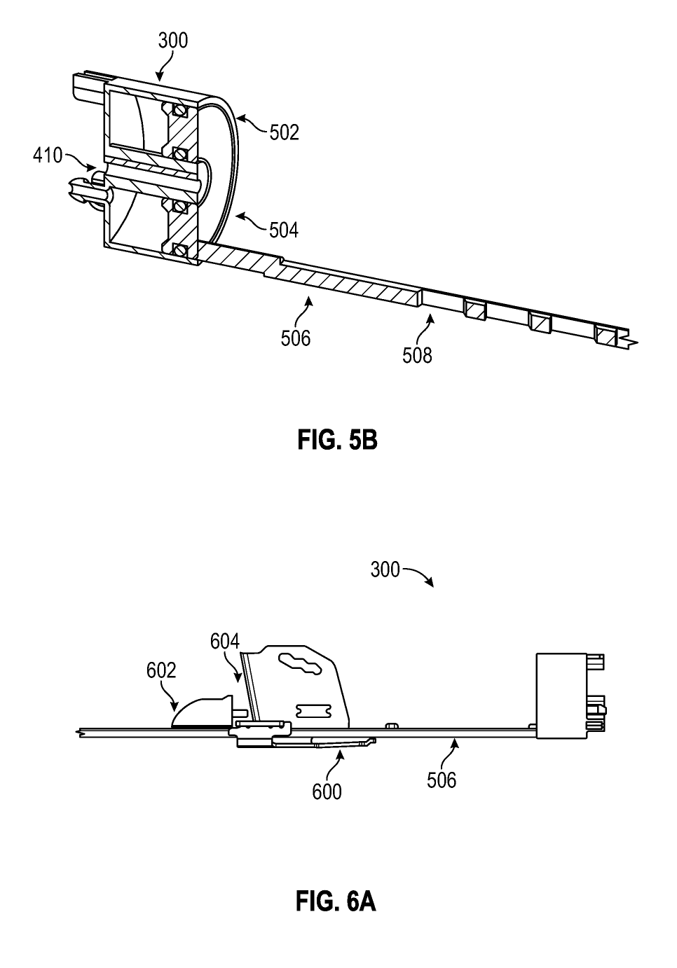

FIG. 5A and FIG. 5B illustrate exposed views of the hydraulic drive system 300. As illustrated in FIG. 5A, the hydraulic drive system 300 includes a piston member 410 contained within a housing member 502. The piston member 410 includes a through channel 504 which provide a pass-through between the proximal portion of the stapling device 100 to the distal portion of the stapling device 100; more particularly, from the shaft member portion 108 to the end-effector portion 110. The piston member 410 is coupled to a drive or actuation member 506. The drive or actuation member 506 is configured to provide the drive force or actuation motion necessary to operate the deployment mechanisms (e.g., staple deployment member 602—such as a wedge member 602—and a cutting member 604—such as a knife member—of the stapling device 100 (located at the end-effector), such as through the reciprocating motion of the piston member 410. Further illustrated in FIG. 5A, the piston member 510 may be positioned in a first position, an initial position or a recovery position in the reciprocating drive cycle of the hydraulic drive system 300. In comparison, FIG. 5B illustrates the piston member 510 in a second piston, an ending position, a power position or a drive position in the reciprocating drive cycle of the hydraulic drive system 300. Also, FIG. 5A and FIG. 5B illustrate that the drive or actuation member 506 includes engagement elements 508 configured to engage with deployment assembly member 600 to drive the deployment operations of the stapling device 100. As may be appreciated, the engagement element 508 may be configured to allow “slip” engagement with the deployment assembly member 600. Such that, during the power portion of the drive cycle, e.g., forward motion of the actuation member 506, the engagement elements 508 allows engagement with the deployment assembly member 600 and drive the deployment assembly member 600 forward for deployment operations, e.g., deployment of staples and deployment of a cutting member. Subsequently, during the recovery portion of the drive cycle, e.g., backward motion of the actuation member 506, the engagement element 508 allows disengagement with the deployment assembly member 600 so that the deployment assembly member 600 momentary remains stationary during the recovery phase of the drive cycle. The hydraulic drive system 300 may operate at such a frequency that the momentary pause of advancement of the deployment assembly member 600 is substantially negligible.

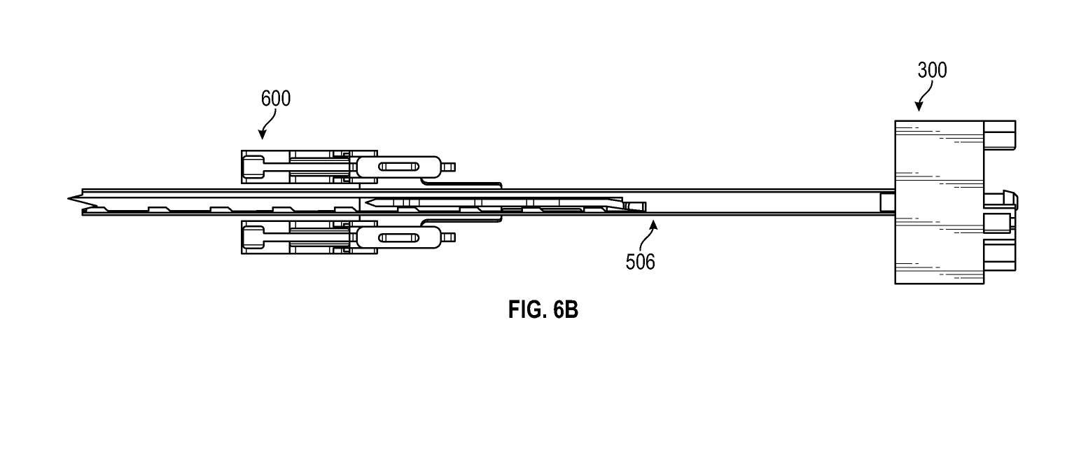

FIG. 6A illustrates a side view that the hydraulic drive system 300 through the drive or actuation member 506 is coupled to a knife member 604 and a wedge assembly 602 of the deployment assembly member 600, wherein the drive or actuation member 506 is configured to advance distally said knife member 604 and said wedge assembly 602 to deploy staples and cut tissue, in accordance with features, aspects or embodiments of the present invention. FIG. 6B illustrates a top view that the hydraulic drive system 300 through the drive or actuation member 506 is coupled to a knife 604 member and a wedge assembly 602, wherein the drive or actuation member 506 is configured to advance distally said knife member 604 and said wedge assembly 602 to deploy staples and cut tissue, in accordance with features, aspects or embodiments of the present invention.

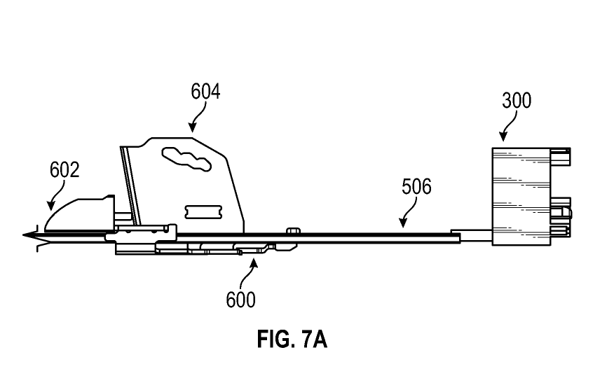

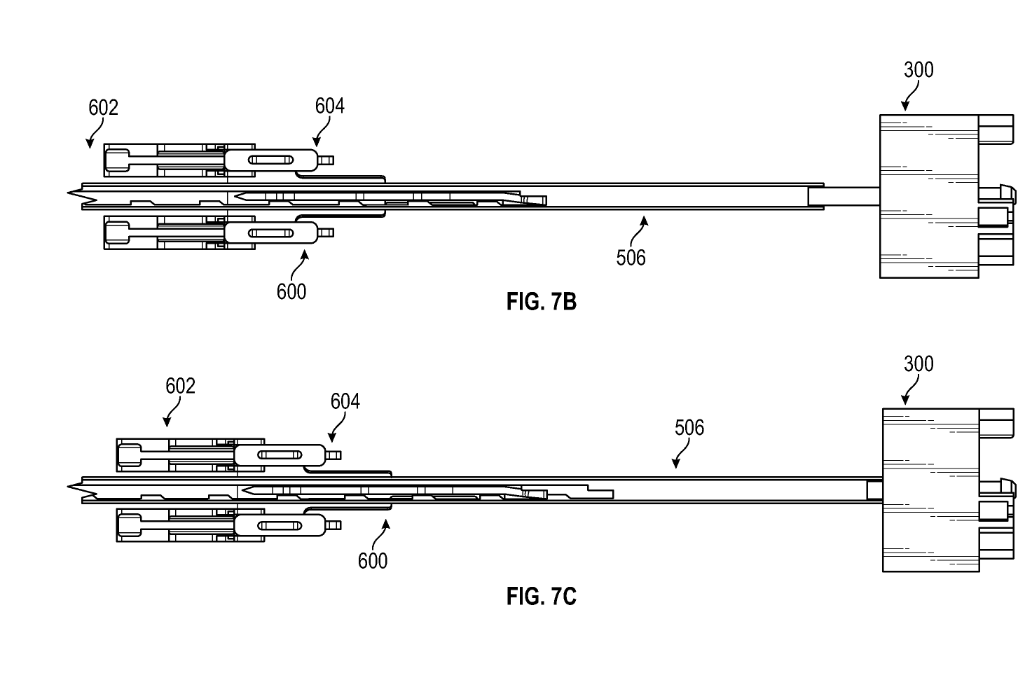

FIG. 7A illustrates the advancement of the drive or actuation member 506 coupled to hydraulic drive system 300 which advances distally the wedge assembly 602 and knife member 604 of the surgical stapling device 100 to deploy staples and cut tissue, in accordance with features, aspects or embodiments of the present invention. FIGS. 7B and 7C illustrate top views the reciprocating movement of the drive or actuation member 506 driven by the piston member 410 to advance distally the wedge assembly 602 and knife member 604 of the surgical stapling device to deploy staples and cut tissue, in accordance with features, aspects or embodiments of the present invention.

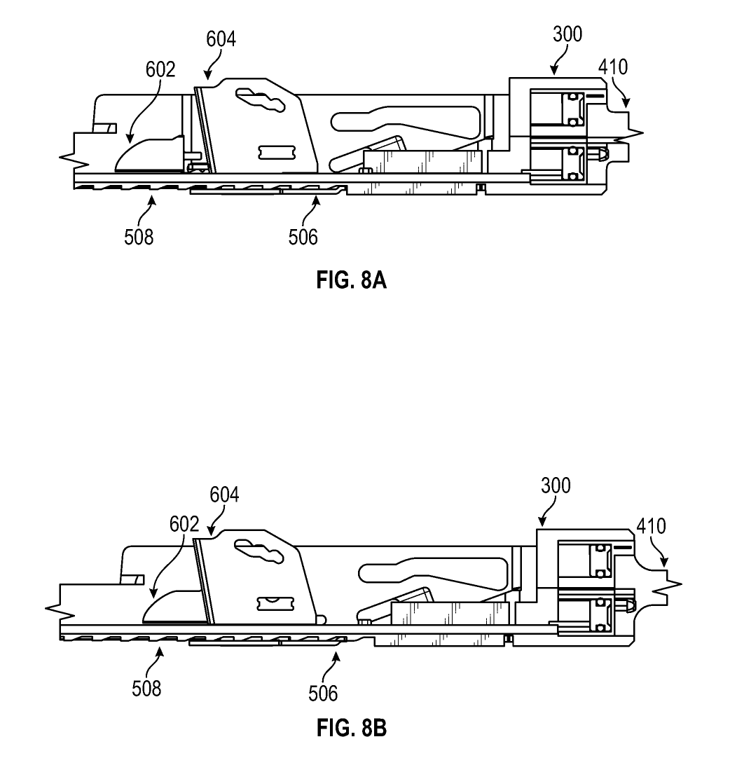

FIGS. 8A and 8B illustrate side views of the reciprocating movement of the drive or actuation member 506 driven by the piston member 410 to advance distally the wedge assembly 602 and knife member 604 of the surgical stapling device 100 to deploy staples and cut tissue, in accordance with features, aspects or embodiments of the present invention.

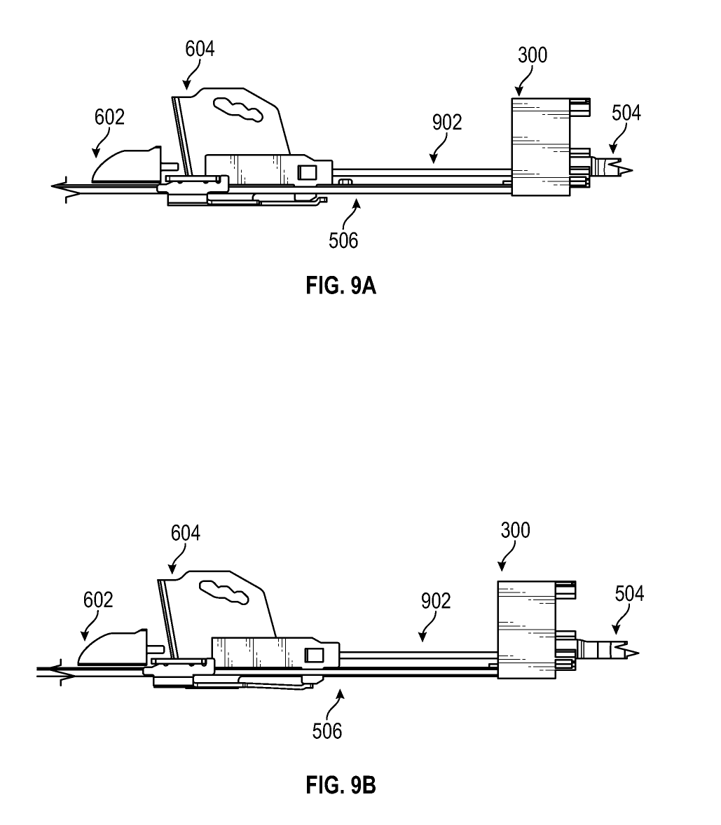

FIG. 9A illustrates the wedge assembly 602 and the knife member 604 being retracted proximally to a first retracted position by a control element 902, wherein the control element 902 is operated through a channel 504 in the hydraulic drive system 300 by control mechanisms in the stapling device, in accordance with features, aspects or embodiments of the present invention. FIG. 9B illustrates the wedge assembly 602 and the knife member 604 of the deployment assembly member 600 being retracted proximally to a second retracted position by a control element 902, wherein the control element 902 is operated through a channel 504 in the hydraulic drive system 300 by control mechanisms in the stapling device 100, in accordance with features, aspects or embodiments of the present invention.

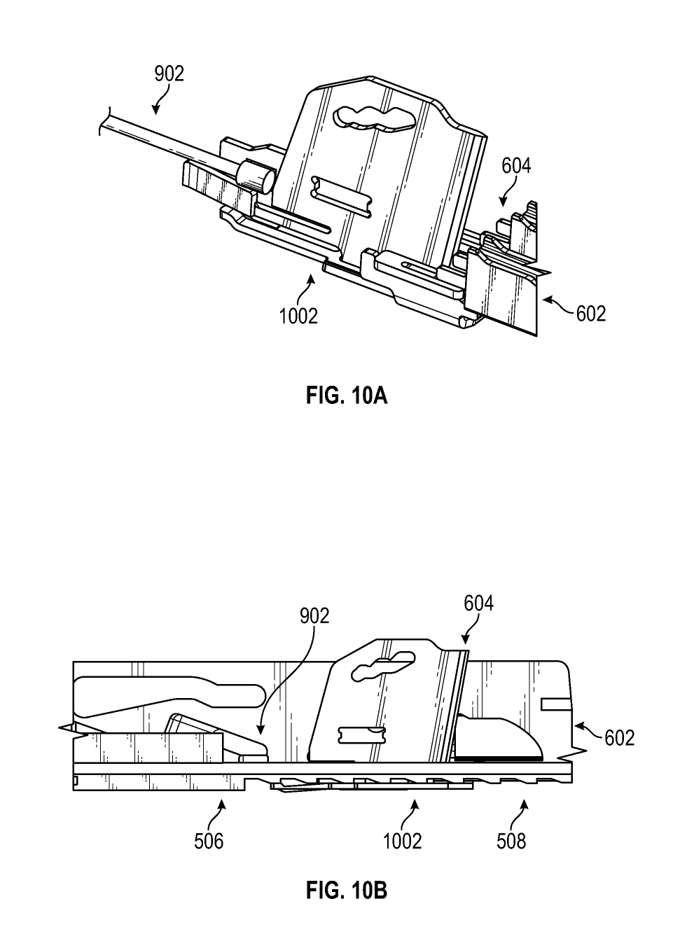

FIG. 10A illustrates a close-up isometric view of the knife member 604 and wedge assembly 602 of the deployment assembly member 600 being retracted proximally by the control element 902, wherein a locking element 1002 allows the knife member and wedge assembly to be retracted proximally along the drive or actuation member 506 and disengaging with engagement elements 508, wherein the drive or actuation member 506 includes features 508 configured to engage the locking element when the drive or actuation member is operated to advance the knife and wedge assembly and the engagement features 508 disengage with the locking element when the knife member 604 and wedge member 602 are retracted, in accordance with features, aspects or embodiments of the present invention. FIG. 10B illustrates a close-up side view of the knife member 604 and wedge assembly 602 being retracted proximally by the control element 902, wherein a locking element 1002 allows the knife member and wedge assembly to be retracted proximally along the drive or actuation member 506, wherein the drive or actuation member 506 includes engagement features 508 configured to engage the locking element 1002 when the drive or actuation member 506 is operated to advance the knife and wedge assembly and the features 508 disengage with the locking element when the knife member and wedge member are retracted, in accordance with features, aspects or embodiments of the present invention.

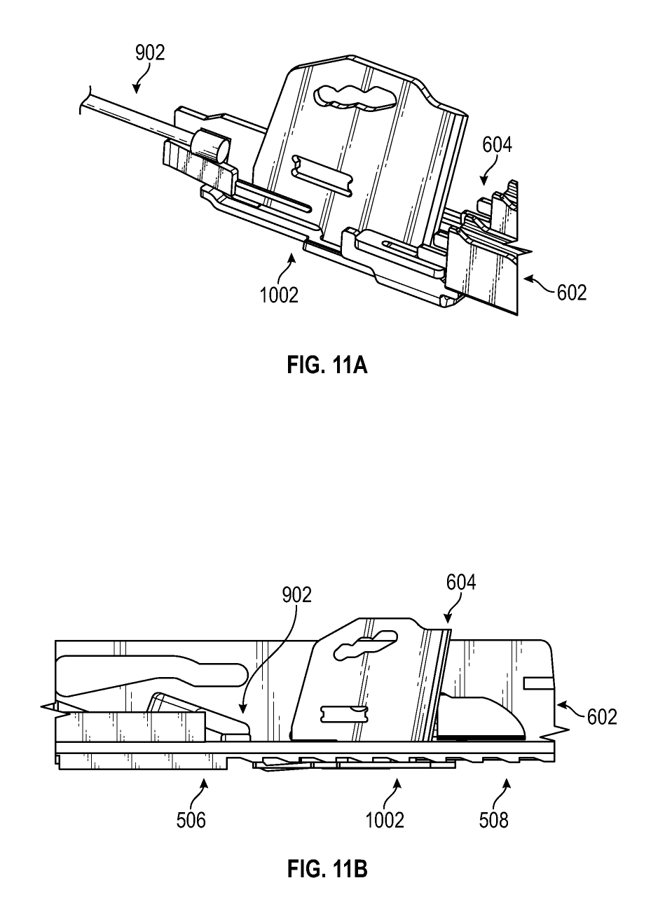

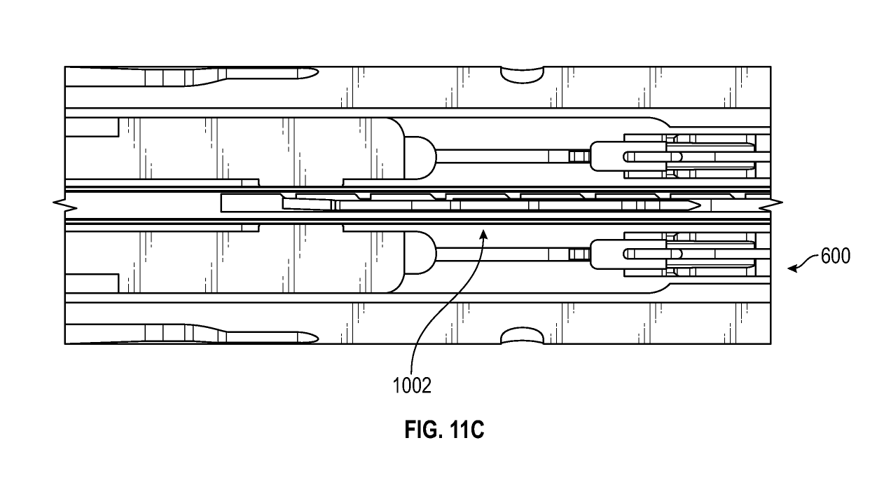

FIG. 11A illustrates another close-up isometric view of the knife member 604 and wedge assembly 602 being retracted proximally by the control element 902, wherein a locking element 1002 allows the knife member 604 and wedge assembly 602 of the deployment assembly member 600 to be retracted proximally along the drive or actuation member 506, wherein the drive or actuation member 506 includes features 508 configured to engage the locking element 1002 when the drive or actuation member 506 is operated to advance the knife member 604 and wedge assembly 602 and the engagement features 508 disengage with the locking element 1002 when the knife member 604 and wedge member 602 of the deployment assembly member 600 are retracted, in accordance with features, aspects or embodiments of the present invention. FIG. 11B illustrates another close-up side view of the knife member 604 and wedge assembly 602 being retracted proximally by the control element 902, wherein a locking element 1002 allows the knife member 604 and wedge assembly 602 to be retracted proximally along the drive or actuation member 506, wherein the drive or actuation member 506 includes engagement features 508 configured to engage the locking element 1002 when the drive or actuation member 506 is operated to advance the knife 604 and wedge assembly 602 and the features 508 disengage with the locking element 1002 when the knife member 604 and wedge member 602 are retracted, in accordance with features, aspects or embodiments of the present invention. FIG. 11C illustrates a top view of the knife member 604 and wedge assembly 602 being retracted proximally by the control element 902, wherein a locking element 1002 allows the knife member 604 and wedge assembly 602 to be retracted proximally along the drive or actuation member 506, wherein the drive or actuation member 506 includes engagement features 508 configured to engage the locking element 1002 when the drive or actuation member 506 is operated to advance the knife and wedge assembly and the features 508 disengage with the locking element 1002 when the knife member 604 and wedge 602 are retracted, in accordance with features, aspects or embodiments of the present invention.

Multiple features, aspects, and embodiments of the invention have been disclosed and described by the illustrated figures. Many combinations and permutations of the disclosed invention may be useful in operating a surgical stapling device, and the invention may be configured to support various surgical procedures. One of ordinary skill in the art having the benefit of this disclosure would appreciate that the foregoing illustrated and described features, aspects, and embodiments of the invention may be modified or altered, and it should be understood that the invention generally, as well as the specific features, aspects, and embodiments described herein, are not limited to the particular forms or methods disclosed, but also cover all modifications, equivalents and alternatives. Further, the various features and aspects of the illustrated embodiments may be incorporated into other embodiments, even if not so described herein, as will be apparent to those ordinary skilled in the art having the benefit of this disclosure.

Although particular features, aspects, and embodiments of the present invention have been illustrated and described, it should be understood that the above disclosure is not intended to limit the present invention to these features, aspects, and embodiments. It will be obvious to those skilled in the art that various changes and modifications may be made without departing from the spirit and scope of the present invention. Thus, the present invention is intended to cover alternatives, modifications, and equivalents that may fall within the spirit and scope of the following claims and their equivalents.

What is claimed:

1. A stapling device, comprising:
an end-effector with an upper jaw member and a lower jaw member;
a deployment assembly member within the end-effector for deploying staples; and
a reciprocating hydraulic drive system within the end-effector to provide direct driving of the deployment assembly to deploy the staples.

2. The stapling device of claim 1 further comprising a shaft member coupled to the end-effector, wherein the shaft member includes a flexible segment to allow articulation of the end-effector,
wherein hydraulic supply lines are routed from within the shaft member to the end-effector to operate the reciprocating hydraulic drive system within the end-effector.

3. The stapling device of claim 1, wherein the reciprocating hydraulic drive system includes a piston member,
wherein the piston member directly drives the deployment assembly member to deploy staples.

4. The stapling device of claim 3 further comprising an actuation member disposed between the piston and the deployment assembly member,
wherein the piston member drives the deployment assembly member through the actuation member.

5. The stapling device of claim 4, wherein the actuation member includes engagement features to engage with the deployment assembly member during a drive stroke of a reciprocating cycle of the reciprocating hydraulic drive system and to disengage with the deployment assembly member during a recovery stroke of the reciprocating cycle of the reciprocating hydraulic drive system.

6. The stapling device of claim 5, wherein the deployment assembly member includes a locking element to engage with at least one of the engagement features of the actuation member during the drive stroke of the reciprocating cycle of the reciprocating hydraulic drive system and to disengage with the at least one of the engagement features of the actuation member during the recovery stroke of the reciprocating cycle of the reciprocating hydraulic drive system.

7. The stapling device of claim 1, wherein the reciprocating hydraulic drive system includes a through-channel to provide a pass-through from the shaft member of the stapling device to the end-effector of the stapling device.

8. The stapling device of claim 7 further comprising a control member originating from the proximal portion of the stapling device disposed through the through-channel to couple with the deployment assembly member, wherein the control member is configured to reset the deployment assembly member.

9. The stapling device of claim 8, wherein the control member resets the deployment assembly member after deployment of staples.
